(19) United States
(12) Patent Application Publication
(10) Pub. No.: US 2014/0211162 A1
(43) Pub. Date: Jul. 31, 2014

(54) OPHTHALMOLOGIC APPARATUS AND METHOD FOR CONTROLLING THE SAME

(71) Applicant: CANON KABUSHIKI KAISHA, Tokyo (JP)

(72) Inventors: Ryutaro Matsuoka, Tokyo (JP); Yukio Sakagawa, Tokyo (JP)

(73) Assignee: CANON KABUSHIKI KAISHA, Tokyo (JP)

(21) Appl. No.: 14/164,002

(22) Filed: Jan. 24, 2014

(30) Foreign Application Priority Data
Jan. 31, 2013 (JP) 2013-017617

(51) Int. Cl. A61B 3/15; A61B 3/12

(52) U.S. Cl. CPC A61B 3/152; A61B 3/12
USPC 351/208

(57) ABSTRACT

An ophthalmologic apparatus includes an imaging unit for imaging the fundus image of a subject's eye, a displacement acquisition unit for acquiring the displacement of an imaging position by the imaging unit between fundus images captured by the imaging unit, and a display control unit for displaying the fundus image captured by the imaging unit and a region of interest on the display unit so that the region of interest is positioned at a predetermined position of the fundus image based on the displacement acquired by the displacement acquisition unit and displaying the region of interest on the display unit by changing a size of the region of interest according to an range where the fundus image is captured.

### BACKGROUND OF THE INVENTION

1. Field of the Invention

The present invention relates to an ophthalmologic apparatus and a method for controlling the ophthalmologic apparatus.

2. Description of the Related Art

An ophthalmic tomography imaging apparatus such as an optical coherence tomography (OCT) is capable of three-dimensionally observing a state inside a retinal layer and useful for more accurately diagnosing disease. Therefore, ophthalmic tomography imaging apparatus has drawn attention in recent years.

Japanese Patent Application Laid-Open No. 2010-227610 discusses a technique for setting an imaging parameter of an OCT tomographic image based on a measurement position specified on a fundus image of a subject's eye. Japanese Patent Publication No. 4262603 discusses a technique for correcting the imaging position of the OCT while a fundus is being tracked to capture the OCT tomographic image because the subject's eye performs an involuntary eye movement during fixation.

The technique discussed in Japanese Patent Application Laid-Open No. 2010-227610 sets a scanner control parameter for manipulating an OCT measurement light based on the position specified on a still fundus image, however, the technique has a problem in that the influence of the involuntary eye movement during fixation is not considered. On the other hand, in a case where the OCT tomographic image is captured while a fundus is being tracked, as discussed in Japanese Patent No. 4262603, the position of a region of interest specified on the fundus image may be different from the position where an actual tomographic image is captured.

### SUMMARY OF THE INVENTION

The present invention is directed to appropriate display of a region of interest on a fundus image.

According to an aspect of the present invention, an ophthalmologic apparatus includes an acquisition unit configured to acquire a fundus image of a subject's eye, a displacement acquisition unit configured to acquire the displacement of an imaging position between the fundus images acquired by the acquisition unit, and a display control unit configured to display on a display unit the fundus image acquired by the acquisition unit and a region of interest which is an area where a tomogram of the fundus is taken, wherein the display control unit displays the fundus image and the region of interest on the display unit based on the displacement acquired by the displacement acquisition unit so that the region of interest is positioned at a predetermined position of the fundus image, and displays the region of interest on the display unit by changing a size of the region of interest according to a range where the fundus image is captured.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is an example illustrating a configuration of an ophthalmologic apparatus according to a first exemplary embodiment.

FIGS. 2A, 2B, and 2C are examples illustrating a configuration of an imaging unit according to the first exemplary embodiment.

FIG. 3 is a flow chart illustrating an example of processing of the ophthalmologic apparatus according to the first exemplary embodiment.

FIGS. 4A and 4B are examples illustrating a display unit of the ophthalmologic apparatus according to the first exemplary embodiment.

FIGS. 5A and 5B are charts for describing the operation of a fundus tracking unit according to the first exemplary embodiment.

FIGS. 6A and 6B are examples illustrating fundus images displayed on a display unit of the ophthalmologic apparatus according to a second exemplary embodiment.

FIG. 7 is a flow chart illustrating an example of processing of the ophthalmologic apparatus according to a third exemplary embodiment.

FIGS. 8A, 8B, 8C, and 8D are examples illustrating a display unit of the ophthalmologic apparatus according to the third exemplary embodiment.

FIGS. 9A, 9B, and 9C are examples illustrating a display unit according to a fifth exemplary embodiment.

FIG. 10 is an example illustrating a display unit according to a sixth exemplary embodiment.

FIG. 11 is examples illustrating fundus images displayed on a display unit according to a seventh exemplary embodiment.

FIG. 12 is an example illustrating a configuration of an ophthalmologic apparatus according to an eighth exemplary embodiment.

FIG. 13 is an example illustrating a configuration of an ophthalmologic apparatus according to a ninth exemplary embodiment.

FIG. 14 is a flow chart illustrating an example of processing of an ophthalmologic apparatus according to a ninth exemplary embodiment.

### DESCRIPTION OF THE EMBODIMENTS

Various exemplary embodiments, features, and aspects of the invention will be described in detail below with reference to the drawings.

A first exemplary embodiment describes a case in which a fundus image and a region of interest indicating the position of a tomographic image are displayed based on information about tracking results. The fundus image and region of interest are displayed when the tomographic image of a fundus is captured while a fundus is being tracked so that the region of interest is positioned in a predetermined position of the fundus image.

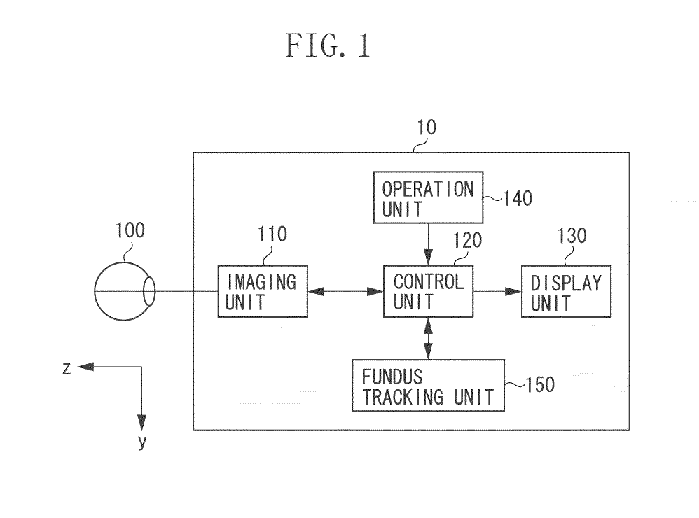

An example of a configuration of an ophthalmologic apparatus 10 according to the first exemplary embodiment is described below with reference to FIG. 1. The ophthalmologic apparatus includes an imaging unit 110, a control unit 120, a display unit 130, an operation unit 140, and a fundus tracking unit 150. The functions of the above units are described in turn below.

[Function of Imaging Unit 110]

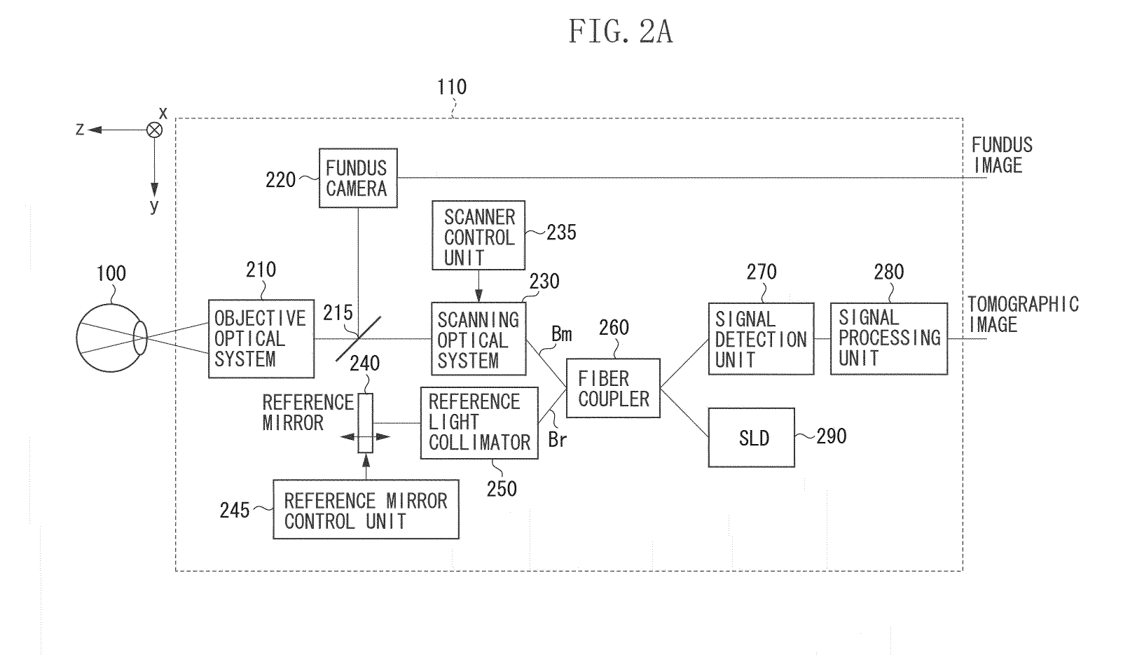

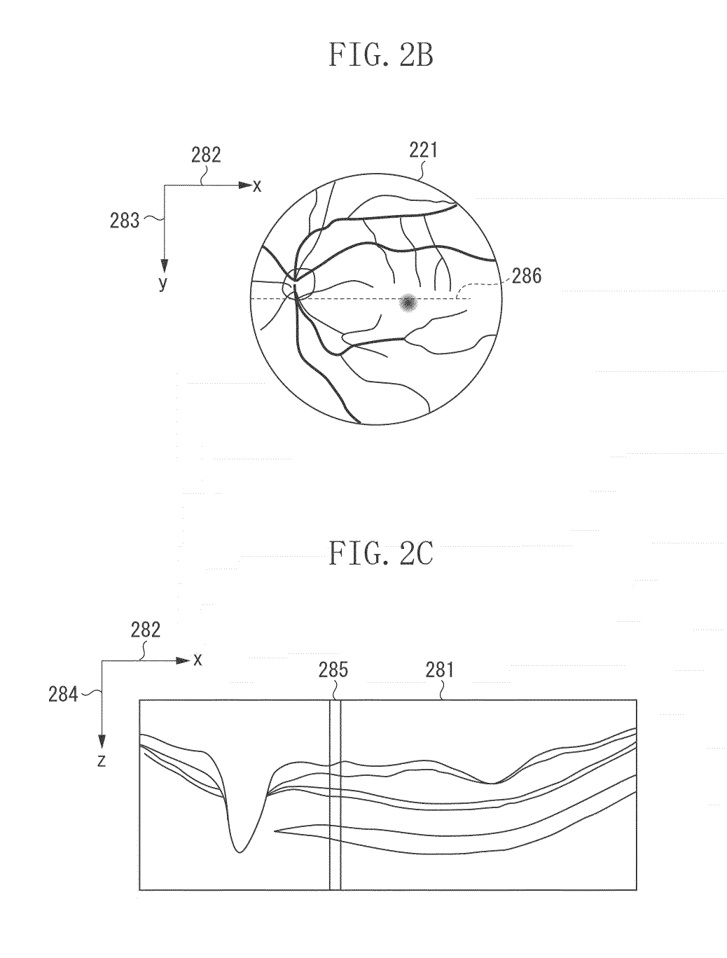

The imaging unit 110 functions as a fundus imaging unit for imaging a two-dimensional image (a fundus image) of a subject's eye 100 or a tomographic imaging unit for imaging the tomographic image of the subject's eye 100. An example of a configuration of the imaging unit 110 is described below with reference to FIG. 2A. The imaging unit 110 includes an objective optical system 210, a half mirror 215, a fundus camera 220, a scanning optical system 230, a scanner control unit 235, a reference mirror 240, a reference mirror control unit 245, a reference light collimator 250, a fiber coupler 260, a signal detection unit 270, a signal processing unit 280, and a super luminescent diode (SLD) 290.

The imaging unit 110 employs a spectral domain system which generates a tomographic image by Fourier-transforming a signal detected by splitting interference light. In FIG. 2A, a direction perpendicular to a drawing paper surface is taken as an X axis and a measurement light scan in the X-axis direction is referred to as a horizontal scan. A downward direction with respect to the drawing paper surface is taken as a Y axis and scan in the Y-axis direction is referred to as a vertical scan.

In FIG. 2A, light emitted from the SLD 290 that is a low-coherence light source is incident on the fiber coupler 260. The fiber coupler 260 separates the incident light into a measurement light Bm and a reference light Br. The measurement light Bm is output to the scanning optical system 230 via an optical fiber. The reference light Br is output to the reference light collimator 250 via the optical fiber.

The scanning optical system 230 condenses the input measurement light Bm into a galvanomirror (not illustrated) to scan with the measurement light. The galvanomirror includes a scanner for horizontal scan and a vertical scanner for vertical scan. The scanner control unit 235 drives and controls both of the scanners. The scanned measurement light Bm reaches a retina of the subject's eye 100 via the objective optical system 210, is reflected by the retina, passes through again the objective optical system 210 and the scanning optical system 230, and reaches the fiber coupler 260. On the other hand, the reference light Br output from the fiber coupler 260 to the reference light collimator 250 is reflected by the reference mirror 240, passes through the reference light collimator 250 again and reaches the fiber coupler 260.

The measurement light Bm and the reference light Br reach the fiber coupler 260 and interfere with each other to generate an interference light. The interference light is output from the fiber coupler 260 to the signal detection unit 270. The reference mirror control unit 245 drives and controls the position of the reference mirror 240. The position of the reference mirror 240 is changed to allow change of the optical path length of the reference light.

The detection unit 270 detects the interference light output from the fiber coupler 260 and outputs an electric interference signal to the signal processing unit 280. The signal processing unit 280 applies Fourier transformation to the interference signal to generate a signal (hereinafter referred to as “A-scan” signal) corresponding to a reflection rate in the Z direction of the retina and acquires the tomographic image of the retina.

The fundus image is captured using the fundus camera 220 and the half mirror 215. Herein, the fundus camera 220 is an infrared camera in the exemplary embodiment, however, the fundus image may also be captured by a confocal scanning laser ophthalmoscope (SLO). A fixation mark is electronically generated by a fixation mark projection unit (not illustrated) and projected onto the retina of the subject's eye 100 to stabilize the fixation. The fixation mark projection unit projects the fixation mark onto the subject's eye 100 based on various parameters such as the projection position, size, shape, and a turned on/off state of light.

Examples of the fundus image and the tomographic image acquired by the imaging unit 110 are described below with reference to FIGS. 2B and 2C. FIG. 2B and FIG. 2C illustrate a fundus image 221 and a tomographic image 281 of the retina respectively. In FIG. 2B and FIG. 2C, an arrow 282 represents the direction of a horizontal scan (X direction), an arrow 283 represents the direction of a vertical scan (Y direction), and an arrow 284 represents the depth direction of the A-scan (Z direction.

The imaging unit 110 causes the signal processing unit 270 to re-structure the A-scan 285 one by one while the scanner control unit 235 moves the galvanomirror of the scanning optical system 230 in a main scanning direction (in this case, in a horizontal direction), so as to form one tomographic image 281. The tomographic image 281 is called a B-scan image which corresponds to a two-dimensional cross section in the depth direction with respect to the retina and the direction orthogonal to the retina, i.e., a plane defined by X and Z axes. A dotted line 286 indicates a position where the tomographic image 281 is captured. The fundus image 221 of the subject's eye 100 is captured by the fundus camera 220.

[Function of Control Unit 120]

The control unit 120 generates imaging control information based on a signal output from the operation unit 140 which receives operation from an inspector. The control unit 120 transfers the imaging control information to the imaging unit 110, and causes the display unit 130 to display various images thereon. A central processing unit (CPU) executes a program stored in a memory (not illustrated) to realize the function of the control unit 120. The control unit 120 includes an imaging control unit 120A and a display control unit 120B which are not illustrated.

The imaging control unit 120A generates an imaging control information according to an operation signal of an operator acquired from the operation unit 140 and outputs the imaging control information to the imaging unit 110. The imaging control unit 120A acquires the fundus image and the tomographic image of the subject's eye 100 from the imaging unit 110. The imaging control information includes information about the imaging position, imaging angle, imaging area of the tomographic image. The imaging position, imaging angle, imaging area of the tomographic image indicate the position and the area where the fundus is scanned with the measurement light to acquire the tomographic image. Those pieces of information are converted into control parameters used when the scanner control unit 235 of the imaging unit 110 controls the scanning optical system 230. Further, the imaging control information may include fixation mark control information for controlling the fixation mark to guide the fixation of the subject's eye 100. The imaging control information may include not only the above information but also the control information of the reference mirror 240 and the focus control information of the objective optical system 210.

The display control unit 120B processes the fundus image and the tomographic image acquired by the imaging control unit 120A and causes the display unit 130 to display the processed images. More specifically, the display control unit 120B superimposes the region of interest onto the fundus image of the subject's eye 100 captured by the imaging unit 110 to generate a synthesized fundus image, according to the imaging control information generated by the imaging control unit 120A and displays the synthesized fundus image on the display unit 130. The region of interest superimposed onto the fundus image refers to the position where the OCT tomographic image is captured and the range where the OCT tomographic image is captured. The region of interest indicated by a line or a frame is displayed on the fundus image. Further, the region of interest indicated by a point, a circle, or a cross may be displayed on the fundus image.

The display control unit 120B also displays and controls a graphic user interface (GUI) for the operator's input operation. The display control unit 120B can be moved according to the instruction of the operation unit 140, for example, and displays an indicator capable of marking any indication position of the display unit 130 on the display unit 130. An arrow cursor, for example, may be used as the indicator, however, the indicator is not limited to the cursor but other different indicators may also be used as long as the indicators can indicate any position of the display unit 130. Thereby, the change of the region of interest can be instructed.

Further, the display control unit 120B can recognize a coordinate on the display unit 130 and recognize the area on the display unit 130 where an indicator exists based on the operation signal input from the operation unit 140. The display control unit 120B can also recognize the coordinate of the area on the display unit 130 where the fundus image is displayed. Accordingly, if the operation unit 140 is a mouse, the display control unit 120B can recognize the position of the indicator on the display unit 130 which moves in response to the movement of the mouse, based on an operation signal indicating the movement of the mouse. Moreover, the display control unit 120B can recognize whether the indicator moving in response to the operation of the operation unit 140 exists in an area on the display unit 130 which displays the fundus image. Furthermore, the display control unit 120B can recognize where the indicator is displayed on the coordinate of the fundus image.

[Function of Display Unit 130]

The display unit 130 displays an image processed by the display control unit 120B, and a GUI layout. The display unit 130 also displays an indicator such as an arrow cursor and other various information.

[Function of Operation Unit 140]

The operation unit 140 outputs an operation signal indicating operation performed by the operator, to the control unit 120 in response to the operation of the operator (not illustrated). Various devices such as a mouse, a keyboard, and a touch panel can be used as the operation unit 140. For example, assume that a mouse equipped with a button and a wheel is used as the operation unit 140. When the mouse serving as the operation unit 140 is pressed for a moment (click), the operation unit 140 outputs an operation signal indicating that the operation unit 140 is clicked, to the control unit 120. When the wheel of the mouse serving as the operation unit 140 is rotated, the operation unit 140 outputs an operation signal indicating a rotation amount of the wheel and an operation signal indicating a rotation direction of the wheel to the control unit 120. Further, when the mouse serving as the operation unit 140 is moved, the operation unit 140 outputs an operation signal indicating movement, to the control unit 120. The operation unit 140 may be composed of a single device such as a mouse or a keyboard, or composed of two or more devices. The operation unit 140 may be composed of a mouse and a keyboard, for example.

[Function of Fundus Tracking Unit 150]

The fundus tracking unit 150 calculates the amount of displacement of the fundus by analyzing the movement of the fundus of the subject's eye 100 from the fundus image captured by the imaging unit 110. In other words, the fundus tracking unit 150 is an example of a displacement acquisition unit for acquiring the displacement of an imaging position between the fundus images acquired by an acquisition unit. If there are a first and a second fundus image captured at two different times, the following processing is performed. The fundus tracking unit 150 sets a region of interest (ROI) 1 On the first fundus image and records the position of the ROI 1. The ROI 1 is a region including an image feature amount such as a strong contrast on the first fundus image. Then, the fundus tracking unit 150 searches for an ROI 2 which is the most correlated with the ROI 1 on the second fundus image. A relative difference between the position of the ROI 1 and the position of the ROI 2 is an amount of displacement of the fundus.

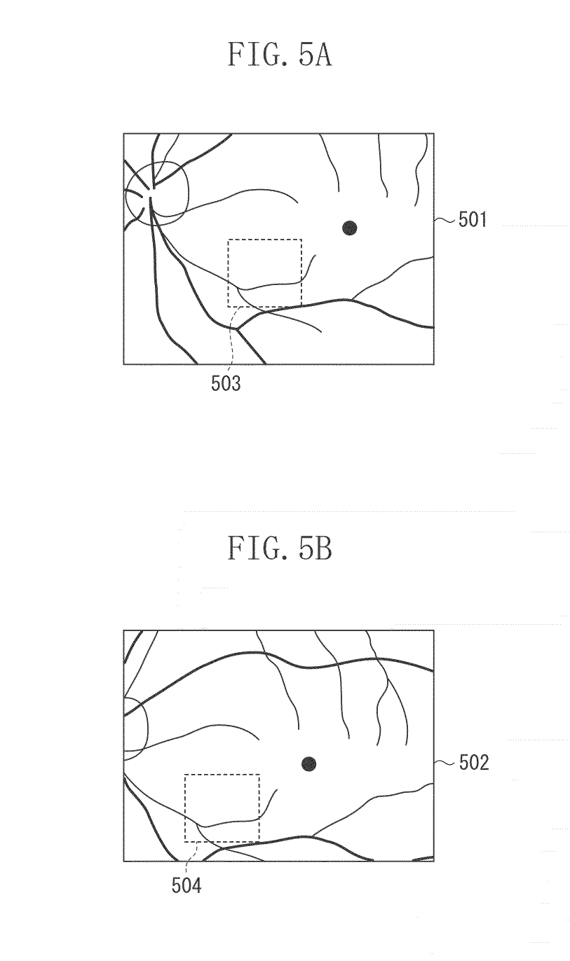

Specific examples are described below with reference to FIGS. 5A and 5B. Fundus images 501 and 502 are images of the same subject's eye 100 captured at two different times. An ROI 503 is set on a fundus image 501 and a search is made in a fundus image 502. As a result, the most correlated ROI 504 is retrieved. If the position of the ROI 503 is (x1, y1) and the position of the ROI 504 is (x2, y2) in the coordinate system of the fundus image, the displacement of two images (dx, dy) is represented by (x2−x1, y2−y1). The position of (x1, y1) may be any position coordinate of the ROI 503, for example, it may be the center coordinate of the ROI 503, or the coordinate of the upper left corner on the drawing paper.

In the present exemplary embodiment, the processing using contrast or correlation is described above, however, any method can be used as long as the amount of a relative displacement between images can be calculated like an optical flow method. Further, two or more ROIs may be set on the fundus image, for example, and the amount of rotation of the fundus may be calculated in addition to the amount of a parallel movement based on the calculation results of the amount of their respective movements.

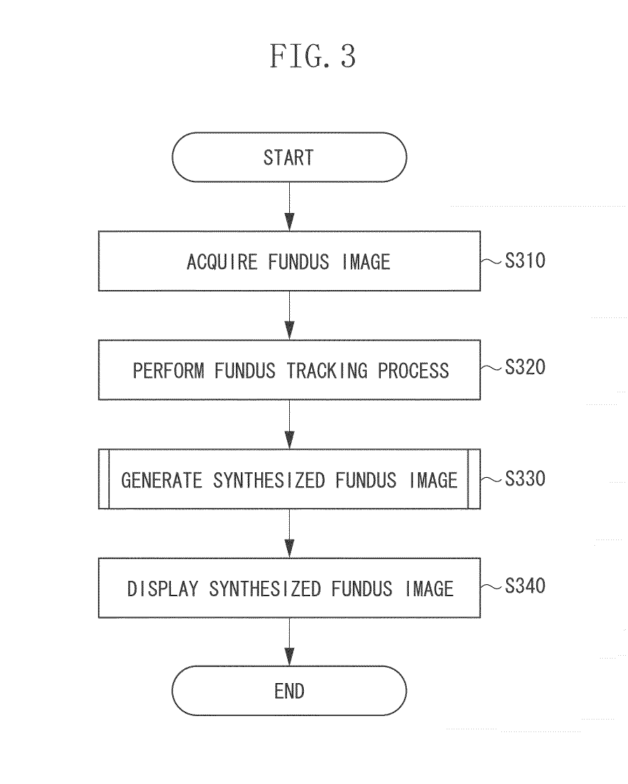

A procedure of a specific processing executed by the ophthalmologic apparatus 10 according to the first exemplary embodiment is described below with reference to a flow chart in FIG. 3.

In step S310, the imaging control unit 120A outputs a command to capture the fundus image, to the imaging unit 110 to acquire the fundus image captured by the imaging unit 110. The imaging control unit 120A outputs the fundus image to the display control unit 120B and the fundus tracking unit 150. Further, the imaging control unit 120A outputs the imaging control information used in capturing the tomographic image, to the display control unit 120B.

In step S320, the fundus tracking unit 150 trucks the fundus to calculate the amount of displacement between the fundus images.

In step S330, the display control unit 120B generates a synthesized fundus image by superimposing the region of interest indicating the position where the tomographic image is captured, on the fundus image. In the present exemplary embodiment, the display control unit 120B generates the synthesized fundus image after correcting the position where a frame indicating the region of interest is superimposed on the fundus image so that the displacement is reduced, based on the amount of displacement of the fundus image calculated in step S320. Resultantly, the region of interest is superimposed on the same region (position) of the sequentially captured fundus image, in other words, on a specific position of the fundus image. More specifically, it is supposed that there are a first and a second fundus image which are captured at different times. The region of interest on the first fundus image is superimposed on the position of (x1, y1), that is, the coordinate of the first fundus image. When the amount of displacement (dx, dy) of the first and the second fundus image is calculated, the region of interest is superimposed on the position of the coordinate (x1+dx, y1+dy) of the second fundus image. In other words, the position indicating the region of interest on the fundus image is moved to perform such control that the region of interest is displayed on a predetermined position.

In step S330, a sideways movement is described as an example, however, if the amount of rotation is also included in the amount of displacement of the fundus image, the position of the region of interest may be corrected using the amount of rotation. Further, the region of interest may be superimposed on the fundus image in a tilted state. Furthermore, as long as the position where the region of interest is displayed can be corrected based on the amount of displacement of the fundus image, other methods may be used for correction.

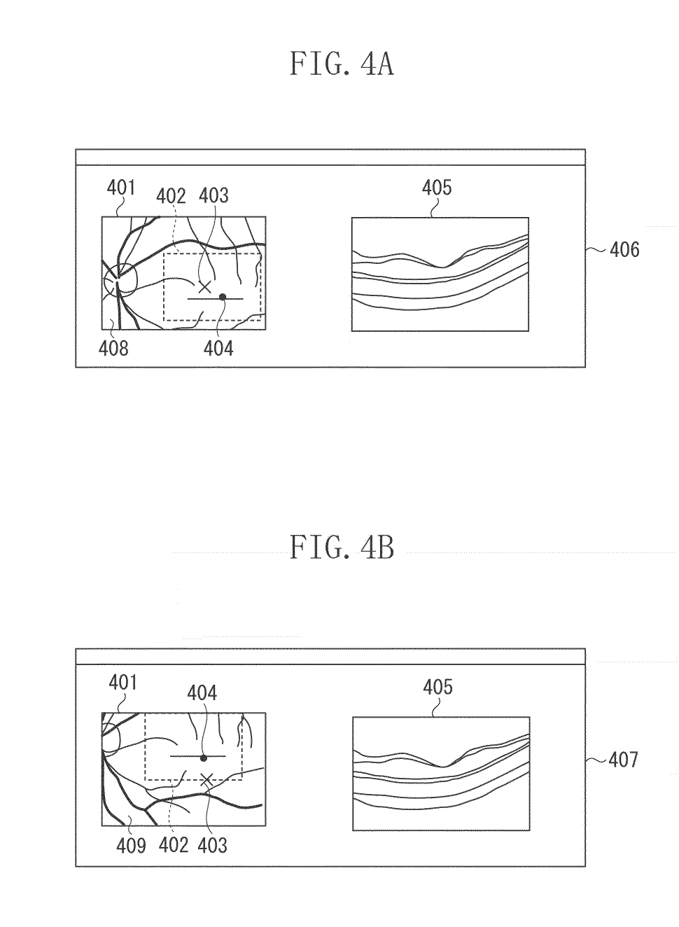

In step S340, the display unit 130 displays the synthesized fundus image generated in step S330. An example of the synthesized fundus image displayed by the display unit 130 according to the first exemplary embodiment is described below with reference to FIGS. 4A and 4B. FIGS. 4A and 4B illustrate display examples 406 and 407 as image display at different times. A fundus image display area 401 is an area for displaying the fundus image and fundus images 408 and 409 are displayed on the fundus image display area 401. A region of interest 402 surrounded by a dotted line indicates a region where the tomographic image is captured. A position 404 where the tomographic image indicated by a line segment is captured is one of image-capture positions in the region of interest 402. The tomographic image 405 is captured in the position 404 where the tomographic image is captured. As illustrated in FIGS. 4A and 4B, as a result of an involuntary eye movement of the subject's eye 100, the fundus images 408 and 409 are located at different positions in the retina. The position where the region of interest is superimposed is corrected on the basis of the tracking result (an amount of movement displacement) between the fundus images 408 and 409, so as to arrange the region of interest 402 at the same region on the fundus. The processing in the flow chart of FIG. 3 is ended here.

As described above, according to the present exemplary embodiment, the position where the region of interest is superimposed is corrected using tracking information between the fundus images (an amount of displacement) when the region of interest indicating the tomographic image capture area and the image capture position is superimposed on the fundus image. In other words, the fundus image and the region of interest indicating the position of the tomographic image are displayed such that the region of interest is placed at a predetermined position of the fundus image. This enables appropriate display of the region of interest on the fundus image while the influence of the involuntary eye movement during fixation is reduced and accurately grasping the position where the tomographic image is captured on the fundus image.

In the first exemplary embodiment, as an example, the position where the tomographic image is captured is accurately grasped by correcting the position where the region of interest (a region indicating the area and the position where the tomographic image is captured) is displayed on the fundus image based on tracking information between the fundus images (an amount of displacement) and by moving the region of interest so as to superimpose and display the region of interest. In a second exemplary embodiment, on the other hand, a method will be described in which the position where the fundus image is displayed is controlled based on the tracking information of the fundus image.

The ophthalmologic apparatus 10 of the second exemplary embodiment is similar in configuration to that of the first exemplary embodiment, so that the description of the configuration is omitted. Processing according to the second exemplary embodiment is similar to the processing illustrated by the flow chart in FIG. 3 according to the first exemplary embodiment except step S330, so that the description thereof is omitted. Step S330 executed in the second exemplary embodiment is described as step S330B.

In step S330B, the display control unit 120B generates a synthesized fundus image by superimposing the area and the position where the tomographic image is captured, on the fundus image. In the present exemplary embodiment, pixels of the fundus image are moved based on the amount of displacement between the fundus images calculated in step S320. More specifically, the amount of displacement between a first and a second fundus image captured at different times is taken as (dx, dy).

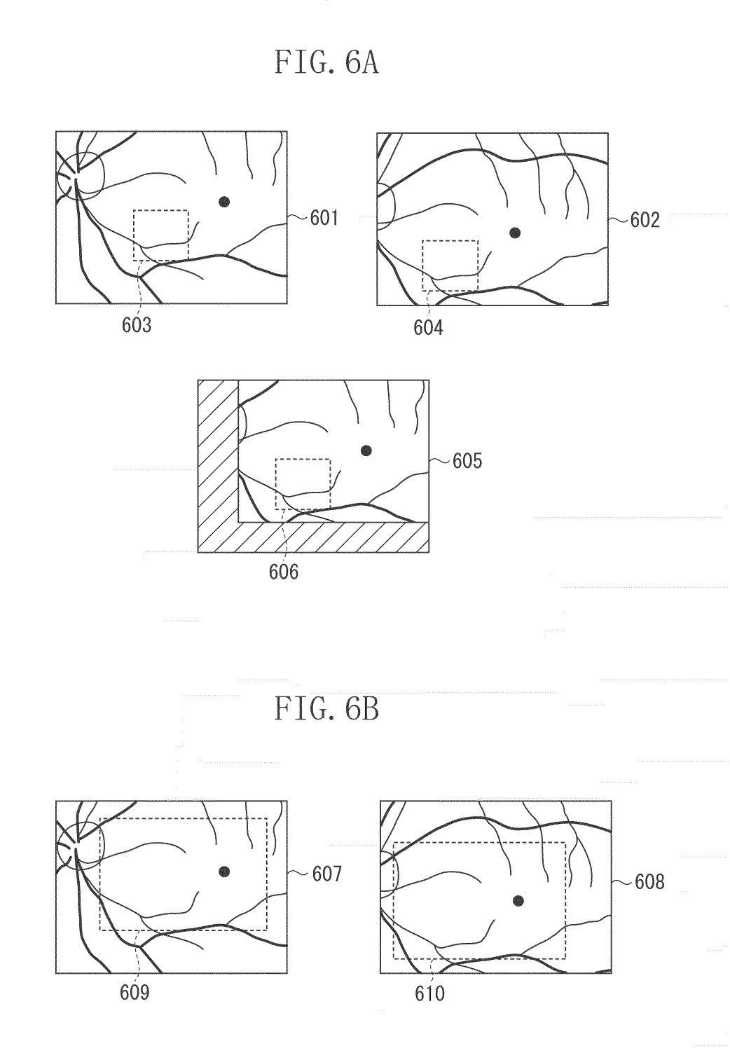

The synthesized fundus image is equal in size to the second fundus image. The synthesized fundus image (x, y) is taken as a second fundus image (x+dx, y+dy) with respect to all pixel positions (x, y) of the synthesized fundus image. The fundus image (x, y) represents the pixel value of the pixel positions (x, y) where the fundus image exists. In other words, with reference to FIG. 6 described below, the pixel value of a fundus image 602 is stored and the pixel value is copied at a position where an amount of displacement is cancelled so as to generate a synthesized image 605.

A specific example is described below with reference to FIG. 6A. Fundus images 601 and 602 are images of the same subject's eye captured at different times. It is assumed that an ROI 603 is set on a fundus image 601 and an area corresponding to the ROI 603 is searched for on a fundus image 602, and as a result, an ROI 604 which is the most correlated with the ROI 603 is found. If the position of the ROI 603 is (x1, y1) and the position of the ROI 604 is (x2, y2) in the coordinate system of the fundus image, the amount of displacement between two pixels (dx, dy) is (x2−x1, y2−y1). As a result of the processing in step S330B, a synthesized fundus image 605 is generated and an ROI 606 indicates the same region as the ROI 604. The synthesized fundus image 605 is generated by moving the fundus image 602 to cancel the amount of displacement (dx, dy). The fundus image 606 in the coordinate system of the synthesized fundus image 605 is equal in position to the ROI 603.

In such processing, an area may occur where a pixel value is unfixed as shown in a shaded area (a left and a lower end of the area) of the synthesized fundus image 605. These pixel values may be expressed in background color such as gray or black, for example, or may be expressed in other colors or by slanted lines. For example, the area where a pixel value is unfixed may have a pixel value at a pixel position same as the fundus image 601. Further, before the processing in step S330B is started, a copy of the first fundus image may be taken as the synthesized fundus image. The pixel value of the fundus image 602 is copied on the copy of the first fundus image based on the amount of displacement, which can eliminate the area where the pixel value is unfixed.

The entire fundus image is moved in step S330B as an example, however, a display area of the fundus image may be clipped. The display control unit 120B may clip a part of the fundus image acquired from the imaging unit 110 and superimpose the region of interest indicating the position where the fundus image is captured on the clipped part. Thus, the possibility that the area of the unfixed pixel value appears is reduced.

More specifically, the first and second fundus images are captured at different times and a display area 1 to be displayed first is set on the first fundus image. The region of interest is superimposed on the image of the display area 1 and output to the display unit 130. Then, the area which is the most correlated with the display area 1 is searched for on the second fundus image and the area is taken as a display area 2. The region of interest is superimposed on the display area 2 and output to the display unit 130.

A specific example is described below with reference to FIG. 6B. Fundus images 607 and 608 are images of the same subject's eye captured at different times. A display area 609 is set on the fundus image 607 and the region of interest superimposed on the display area 609 is displayed on the display unit 130. A display area 610 which is the most correlated with the display area 609 is searched for on the fundus image 608. The region of interest is superimposed on the display area 610 and the region of interest superimposed thereon is displayed on the display unit 130. Thus, areas 609 and 610 which are parts of the fundus image are displayed on the display unit 130, so that the possibility that an area where the pixel value is unfixed appears due to the movement of a subject's eye, can be further reduced as compared with a case where all areas of the captured fundus image are displayed on the display unit 130.

As described above, according to the present exemplary embodiment, the position where the fundus image is displayed is controlled based on the tracking information about the fundus image at the time of superimposing the region of interest indicating the area and the position where the tomographic image is captured, on the fundus image. Accordingly, a less variable fundus image is captured, reducing the motion of the region of interest on the fundus image, so that the position of the range where the tomographic image is captured becomes more comprehensible.

In the first exemplary embodiment, the example is described in which the region of interest is superimposed and displayed on the fundus image to accurately grasp the position where the tomographic image is captured based on tracking information of the fundus image. In a third exemplary embodiment, a display control method for reducing the motion of the region of interest when the operator operates the region of interest is described. The ophthalmologic apparatus 10 of the third exemplary embodiment is similar to that of the first exemplary embodiment in configuration, so that the description thereof is omitted.

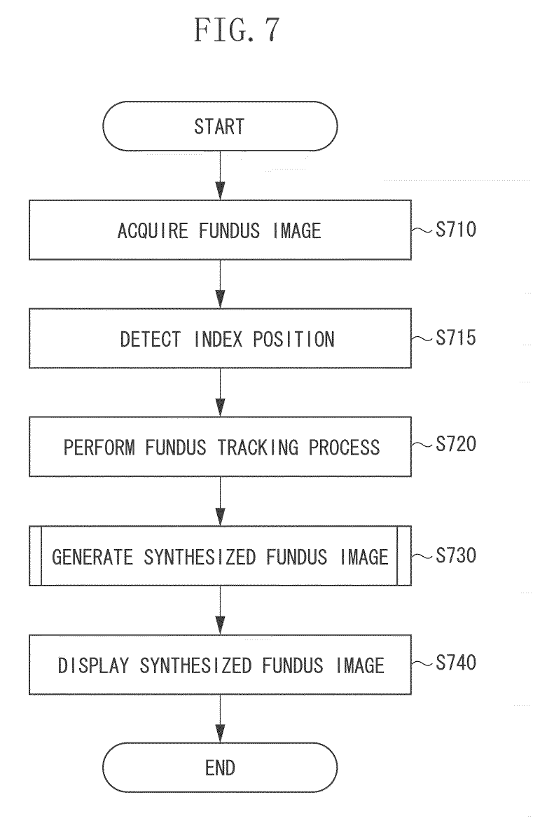

A procedure of a specific processing executed by the ophthalmologic apparatus 10 according to the third exemplary embodiment is described below with reference to a flow chart in FIG. 7. The processing in steps S710, S720, and S740 is similar to steps S310, S320, and S340 respectively, so that the description thereof is omitted.

In step S715, the operation unit 140 outputs an operation signal indicating operation from the operator to the control unit 120.

In step S730, the display control unit 120B generates a synthesized fundus image by superimposing the region of interest indicating the position where the tomographic image is captured, on the fundus image. More specifically, the display control unit 120B acquires the position of an index moving on the display unit 130 according to the instruction of the operation unit 140 to determine whether the index exists on the fundus image displayed on the display unit 130. A method for synthesizing the fundus image is different according to the determination result.

Specifically, the fundus image is displayed on a part of the area of the display unit. If the index indicating any position of the display unit also exists in the area of the display unit except the part of the area, the region of interest is moved on the display unit based on the calculated displacement. On the other hand, if the index indicating any position of the display unit exists on the fundus image, the movement of the region of interest is stopped.

Thus, if the index is not found on the fundus image, the same process as that in step S330 is conducted. If the index is found on the fundus image, on the other hand, the region of interest is superimposed on the same position as the position of the region of interest of the last fundus image, instead of correcting the portion of amount of displacement of the fundus image at the time of superimposing the region of interest on the fundus image. In other words, the movement of the region of interest on the fundus image is stopped. The example described in the above makes a determination depending on whether the index is on the fundus image. However, the determination may be made depending on whether the index is in the region of interest, for example. More specifically, if the index is in the area on the display unit outside the region of interest, the region of interest is moved on the display unit based on the calculated displacement. If the index is in the region of interest, the movement of the region of interest may be stopped.

In the present exemplary embodiment, the region of interest may be operated by the operation signal input from the operation unit 140. For example, when the index is in the region of interest, a mouse click is performed using a mouse functioning as an instruction unit for changing the position where the region of interest is displayed by manipulating the position where the index is displayed. The region of interest may be moved on the fundus image by the mouse grabbing and dragging the region of interest. When the index is on the edge of the region of interest, the region of interest may be grabbed by the mouse to change the size of the region of interest. Information about the changed region of interest is transferred to the imaging control unit 120A. The imaging control unit 120A calculates a position on the fundus relative to the region of interest. The imaging control unit 120A changes imaging control information of the tomographic image of the imaging unit 110 to capture the tomographic image at a position on the fundus image and outputs the information to the control unit 110.

More specifically, if a mouse is used as the operation unit 140, for example, when the index is on the fundus image and click is performed on the fundus image, the display control unit 120B receives an operation signal according to the click. The display control unit 120B calculates a distance between the coordinate position of the index when the click is performed, and a predetermined position of the area where the fundus image is displayed on the display unit 130. A unit of the distance is a pixel, for example. The position where the region of interest is displayed is changed according to the calculation result.

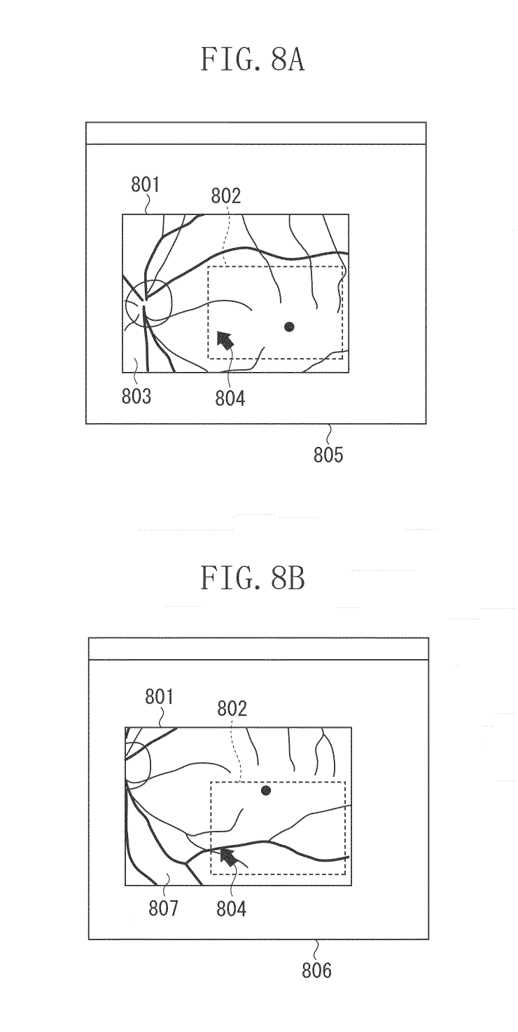

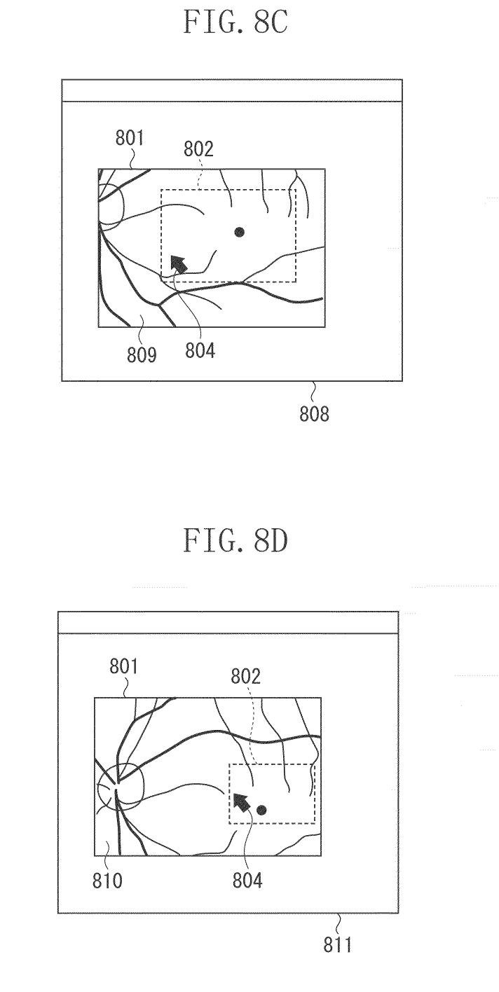

Examples of resulting display in the ophthalmologic apparatus according to the present exemplary embodiment are described below with reference to FIGS. 8A, 8B, 8C, and 8D. FIGS. 8A, 8B, 8C, and 8D illustrate display examples 805, 806, 808, and 811 at different times. A fundus image display area 801 is an area where the fundus image is displayed and fundus images 803, 807, 809, and 810 captured at different times are displayed in FIGS. 8A, 8B, 8C, and 8D. A region of interest 802 indicates a range where the tomographic image is captured. In the display example 805 illustrated in FIG. 8A, an index 804 lies in the region of interest 802, so that the position where the region of interest 802 is superimposed is the same as that in FIG. 8A as indicated by the display example 806 in FIG. 8B. When the mouse is clicked in a state of FIG. 8B, the position of the region of interest 802 is dragged by the mouse and moved (to the upper left direction on the drawing paper) as indicated by the display example 808 in FIG. 8C to change the position where the region of interest 802 is superimposed on the fundus image 809. FIG. 8D illustrates an example in which the size of the region of interest 802 is changed by the operation of the mouse.

As described above, according to the present exemplary embodiment, the position where the region of interest is superimposed on the fundus image is corrected based on the fundus tracking information and the index position. Accordingly, it becomes easy to grasp the position where the tomographic image is captured and manipulate the position and the range where the tomographic image is captured.

In the third exemplary embodiment, the display control method for decreasing the movement of the region of interest when the operator manipulates the region of interest has been disclosed. In a fourth exemplary embodiment, the display control of the fundus image performed when the operator manipulates the region of interest is described.

The ophthalmologic apparatus 10 of the fourth exemplary embodiment is similar in configuration to that of the first exemplary embodiment, so that the description of the configuration is omitted. Processing according to the fourth exemplary embodiment is similar to the processing illustrated by the flow chart in FIG. 7 according to the third exemplary embodiment except step S730, so that the description thereof is omitted. Step S730 executed in the fourth exemplary embodiment is described as step S730B.

In step S730B, the display control unit 120B generates a synthesized fundus image by superimposing the area and the position where the tomographic image is captured, on the fundus image. More specifically, the display control unit 120B acquires the position of an index moving on the display unit 130 according to the instruction of the operation unit 140 to determine whether the index exists on the fundus image displayed on the display unit 130. A method for synthesizing the fundus image is different according to the determination result.

If the index is not on the fundus image, the same process as that in step S330 is conducted. If the index is on the fundus image, on the other hand, the same process as that in step S330B is conducted, in which the region of interest is superimposed on the same position as the position of the region of interest of the last fundus image without consideration of the amount of displacement of the fundus image, at the time of superimposing the region of interest on the fundus image. In other words, the movement of the region of interest on the fundus image is stopped. Although the example described in the above makes a determination depending on whether the index is on the fundus image, the determination may be made depending on whether the index is in the region of interest, for example. As is the case with the third exemplary embodiment, also in the present exemplary embodiment, the region of interest may be manipulated by the operation signal input from the operation unit 140.

As described above, according to the present exemplary embodiment, the method for displaying the fundus image is corrected based on the fundus tracking information and the index position. Accordingly, it becomes easy to grasp the position where the tomographic image is captured, and manipulate the position and the range where the tomographic image is captured. The position where the tomographic image is captured is less frequently changed on the fundus image even in the manipulation of the region of interest, so that the position where the tomographic image is captured on the fundus image can be more accurately specified.

In the fifth exemplary embodiment, an example will be described in which the area where the fundus image is displayed is changed into a size according to the range where the fundus image is captured, when the distance between the imaging unit 110 and the subject's eye 100 is changed.

The ophthalmologic apparatus 10 of the present exemplary embodiment is substantially similar in configuration to that of the first exemplary embodiment, so that the description of the configuration is omitted. The processing of the present exemplary embodiment is also substantially similar to that of the first exemplary embodiment, so that the description thereof is omitted.

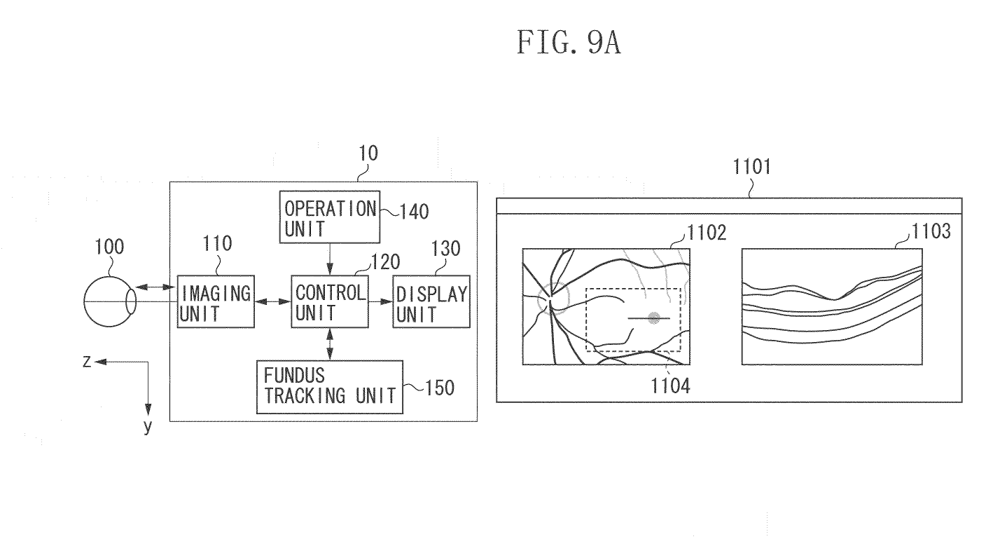

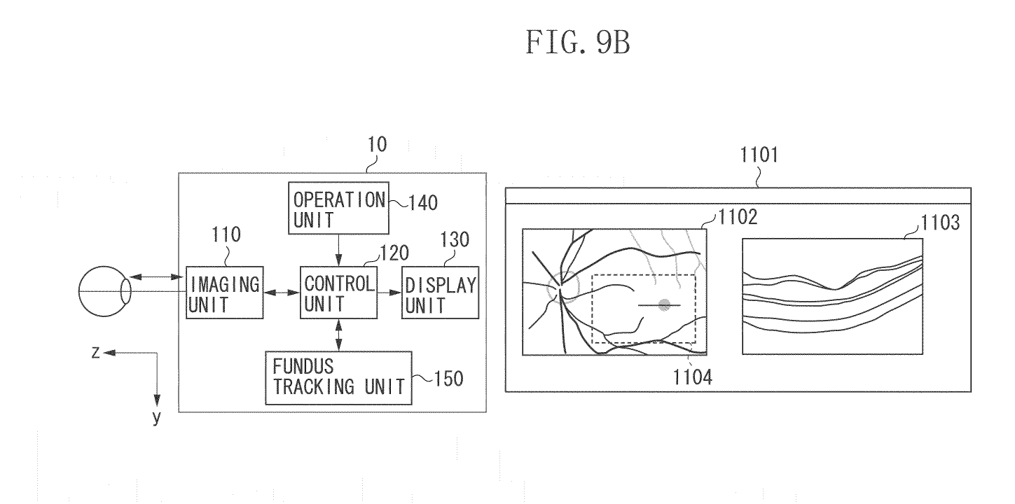

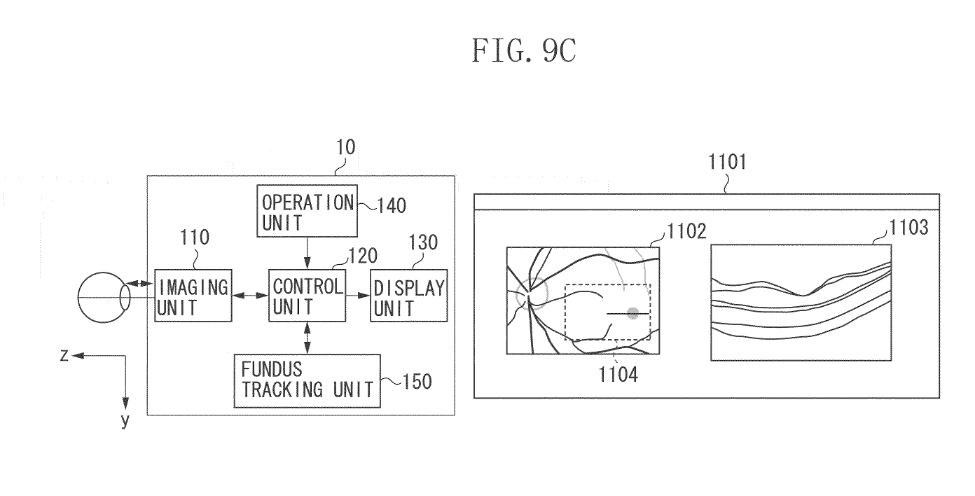

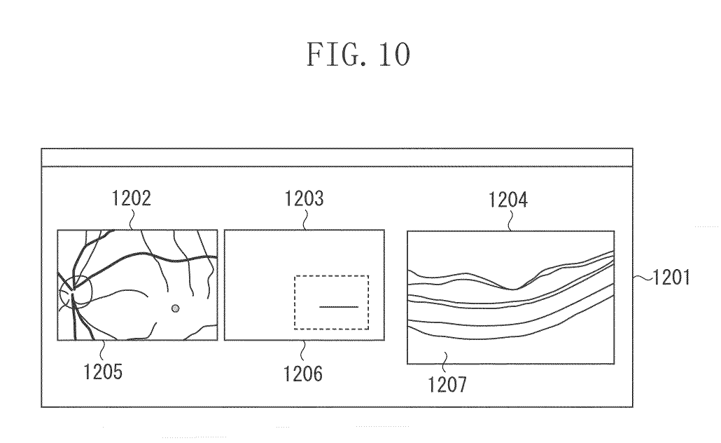

Examples displayed by the display unit 130 according to the present exemplary embodiment are described with reference to FIGS. 9A, 9B, and 9C.

An angle of view that is a range where the fundus image is captured is determined according to a distance between the objective optical system 210 of the imaging unit 110 and the subject's eye 100 in the depth direction (Z direction). In other words, if the imaging unit 110 is moved to push the objective optical system 210 away from the subject's eye 100, the range where the fundus image is captured is enlarged. On the other hand, if the imaging unit 110 is moved to bring the objective optical system 210 closer to the subject's eye 100, the range where the fundus image is captured is decreased. The display control unit 120B recognizes the range where the fundus image is captured, changes the area where the fundus image is displayed according to the size of the range where the fundus image is captured and displays the area on the display unit 130.

FIG. 9B illustrates an example in which the imaging unit 110 is moved away from the subject's eye 100 in FIG. 9A. In this case, the acquired fundus image is enlarged, so that the fundus image is displayed with the increased size of a fundus image display area 1102. In other words, the display control unit 120B which is an example of a display control unit changes the size of the region of interest according to the range where the fundus image is captured and displays the fundus image on the display unit.

On the other hand, FIG. 9C illustrates an example in which the imaging unit 110 is brought closer to the subject's eye 100 in FIG. 9A. In this case, the acquired fundus image is reduced, so that the fundus image is displayed by decreasing a size of a fundus image display area 1102 and the region of interest. In other words, in a case where the size of the fundus image display range where the fundus image is displayed on the display unit is changeable according to a range where the fundus image is captured, the greater the range where the fundus image is captured, the greater the fundus image display area and the region of interest.

As for a magnification used in enlarging and reducing the fundus image display area 1102, the range where the fundus image is captured is defined as a reference, for example, and the magnification may be equalized with that of the acquired range where the fundus image is captured with respect to a reference range where the fundus image is captured.

Alternatively, the size of the fundus image display area 1102 may not be changed and the acquired fundus image may be enlarged or reduced according to the size of the fundus image display area 1102. For example, the range where the fundus image is captured is larger in FIG. 9B than that in FIG. 9A, so that the fundus image is reduced and displayed in the fundus image display area 1102. Therefore, the region of interest 1104 is also reduced and displayed. On the other hand, the range where the fundus image is captured is smaller in FIG. 9C than that in FIG. 9A, so that the fundus image is enlarged and displayed in the fundus image display area 1102. Therefore, the region of interest 1104 is also enlarged and displayed. In other words, in a case where the size of the fundus image display area where the fundus image is displayed on the display unit is fixed, the greater the range where the fundus image is captured, the smaller the region of interest.

If the fundus image is captured by scanning with the light of the SLO, the pivot position of scanned light is positioned in the pupil of the subject's eye 100 to prevent shading light from arising due to iris or lash. Therefore, a light scanning range in FIG. 9B is narrower than the light scanning range in FIG. 9A to prevent shading light due to iris or lash. Thereby, the fundus image capture range acquired in FIG. 9B is smaller than the fundus image capture range acquired in FIG. 9A.

For the similar reason, a light scanning range in FIG. 9C becomes wider than the light scanning range in FIG. 9A to prevent shading light from arising due to iris or lash. Thereby, the fundus image capture range acquired in FIG. 9C is larger than the fundus image capture range acquired in FIG. 9A.

Thus, the processing for enlarging or reducing the fundus image display area 1102 and the region of interest 1104 can be applied also when the shading light due to iris or lash is taken into consideration. The exemplary embodiment can also be applied in which the acquired fundus image is enlarged or reduced according to the size of the fundus image display area 1102 without changing the size of the fundus image display area 1102.

If the distance between the imaging unit 110 and the subject's eye 100 in the Z direction is changed, the optical path length of the measurement light Bm is changed and the desired interference light is not generated between the measurement light Bm and the reference light Br. The reference mirror control unit 245 moves the reference mirror 240 by a predetermined amount to change the optical path length of the reference light Br and performs control to acquire the desired interference light.

As described above, according to the present exemplary embodiment, even if the distance between the imaging unit 110 and the subject's eye 100 is changed, the fundus image and the region of interest is displayed according to the range where the fundus image is captured so as to enable more accurate grasping of the position where the tomographic image is captured on the fundus image.

The first exemplary embodiment describes the region of interest which is superimposed on the fundus image of the subject's eye. A sixth exemplary embodiment will describe a configuration in which an area where the region of interest is displayed is provided separately from the area where the fundus image is displayed.

The ophthalmologic apparatus 10 of the present exemplary embodiment is substantially similar to that of the first exemplary embodiment in configuration, so that the description thereof is omitted. The processing according to the present exemplary embodiment is also substantially similar to the processing according to the first exemplary embodiment, so that the description thereof is omitted.

An example displayed by the display unit 130 according to the present exemplary embodiment is described with reference to FIG. 10.

In a display example 1201, there are provided a fundus image display area 1202, a region-of-interest display area 1203, and a tomographic image display area 1204. The display control unit 120B displays a fundus image 1205 and a tomographic image 1207 acquired by the imaging control unit 120A in the fundus image display area 1202 and the tomographic image display area 1204 respectively. The display control unit 120B displays the position corresponding to the range where the tomographic image is captured on the fundus image 1205 as a region of interest 1206 in the region-of-interest display area 1203 with a line and a frame. As described in the above exemplary embodiment, the display control unit 120B controls the position where the region of interest is displayed within the region-of-interest display area 1203 on the basis of the movement of the subject's eye. Alternatively, the display control unit 120B displays the fundus image in the fundus image display area 1202 to reduce the movement of the fundus image according to the movement of the subject's eye while the display of the region of interest remains fixed in the region-of-interest display area 1203. In other words, the display control unit 120B causes the display unit to display the fundus image and the region of interest such that a positional relationship is retained between the fundus image and the region of interest, on the basis of the displacement acquired by the displacement acquisition unit.

The region-of-interest display area 1203 is made equal in size to the fundus image display area 1202 so that the position of the region of interest 1206 on the fundus image can be more easily grasped. The region-of-interest display area 1203 is reduced and the fundus image is previewed as a background in the area 1203 so that the position of the region of interest 1206 can be easily grasped.

As described above, according to the present exemplary embodiment, the region of interest is displayed in another display area without being superimposed on the fundus image so that the fundus image can be clearly identified and the position where the tomographic image is captured can be easily grasped.

The second exemplary embodiment describes the method for controlling the position where the fundus image is displayed based on the tracking information of the fundus image. In that case, as an example, the area where a pixel value of a synthesized fundus image is unfixed is expressed by a background color or slanted lines, or is implanted with the same pixel value as that of the original fundus image. In a seventh exemplary embodiment, on the other hand, an example will be described in which the synthesized fundus image is enlarged and displayed to decrease the area where a pixel value is unfixed.

The ophthalmologic apparatus 10 of the present exemplary embodiment is substantially similar to that of the first exemplary embodiment in configuration, so that the description thereof is omitted. The processing according to the present exemplary embodiment is also substantially similar to the processing according to the second exemplary embodiment, so that the description thereof is omitted.

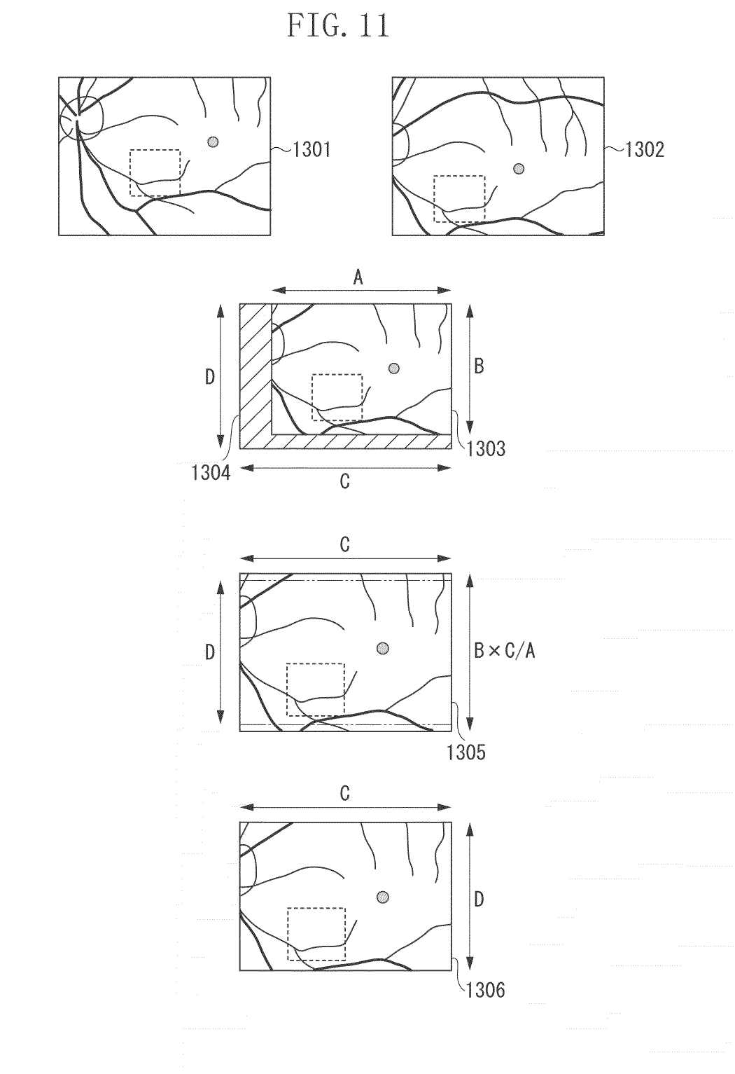

A specific example of a synthesized fundus image according to the present exemplary embodiment is described below with reference to FIG. 11.

Fundus images 1301 and 1302 are the same subject's eye 100 captured at different times. The two fundus images are subjected to image processing using the same method as that in the second exemplary embodiment to generate a synthesized fundus image 1303. The synthesized fundus image 1303 corresponds to a common portion between the fundus images. If an area where a pixel value is unfixed arises in a part of the fundus image display area are, for example, a left or a lower end area, the display control unit 120B displays the enlarged fundus image on the display unit 130 to minimize the area where the pixel value is unfixed.

If the image size of the synthesized fundus image 1303 is of A (horizontal)×B (vertical) and the size of a fundus image display area 1304 is of C (horizon)×D (vertical), for example, the display control unit 120B generates an enlarged fundus image 1305 in which the synthesized fundus image 1303 is enlarged by a magnification of C/A or D/B, whichever is greater. Then, the display control unit 120B generates an enlarged fundus image 1306 in which a portion protruding from the fundus image display area 1304 in the enlarged fundus image 1305 is deleted. More specifically, if the synthesized fundus image 1303 is enlarged by a magnification of C/A, the size of the enlarged fundus image 1305 is C (horizontal)×[B×C/A] (vertical) so as to generate the enlarged fundus image 1306 in which a portion protruding from the fundus image display area 1304 [B×C/A−D] is deleted. If the synthesized fundus image 1303 is enlarged by a magnification of D/B, the display control unit 120B generates an enlarged fundus image 1306 in which [A×D/B−C] (horizontal) is deleted from the enlarged image. In other words, the common portion and the region of interest are enlarged and displayed.

The area where the pixel value is unfixed in the fundus image display area can be minimized by displaying the fundus image 1306 enlarged by the above method in the fundus image display area 1304 of the display unit 130.

In the above description of the example, the fundus image is enlarged with the aspect ratio of the original image maintained, however, the present exemplary embodiment is not limited to the above. The left and the right magnification may be changed so that the synthesized fundus image can be displayed all over the fundus image display area.

As described above, according to the present exemplary embodiment, the synthesized fundus image is enlarged and displayed such that the area where the number of pixels is unfixed can be reduced when the position for displaying the fundus image is controlled based on the tracking information of the fundus image.

The first present exemplary embodiment has described a configuration in which the fundus image of the subject's eye 100 is captured using the fundus camera 220 incorporated in the imaging unit 110. An eighth exemplary embodiment will describe a configuration in which the fundus image is generated in a pseudo manner on the basis of the signal information of the tomographic image of the subject's eye.

The ophthalmologic apparatus 10 of the present exemplary embodiment is substantially similar to that of the first exemplary embodiment in configuration, so that the description thereof is omitted. The processing according to the present exemplary embodiment is also substantially similar to the processing according to the first exemplary embodiment, so that the description thereof is omitted.

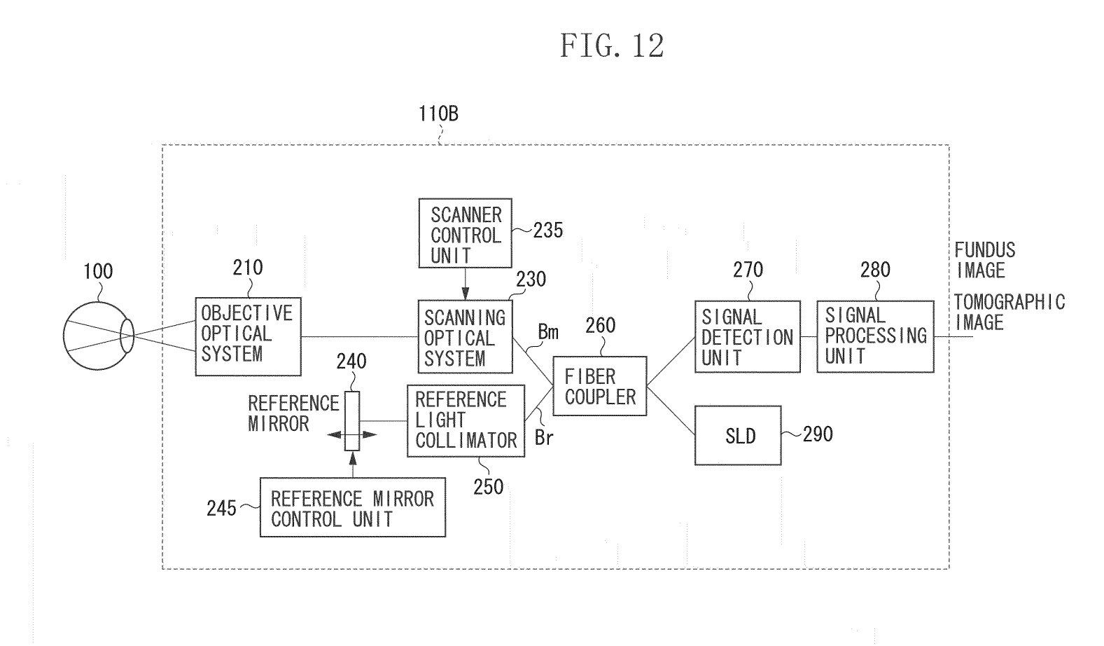

A specific example of an imaging unit 110B according to the present exemplary embodiment is described below with reference to FIG. 12.

The imaging unit 110B according to the present exemplary embodiment has a configuration in which the half mirror 215 and the fundus camera 220 of the imaging unit 110 according to the first exemplary embodiment are removed, but functions of the components are substantially similar to those of the first exemplary embodiment.

In the present exemplary embodiment, the signal processing unit 280 applies signal processing to an interference signal between the measurement light Bm and the reference light Br to acquire the tomographic image of the fundus image, generates the pseudo image of the fundus image, and outputs the pseudo image to the imaging control unit 120A. In other words, the fundus image acquired based on the interference light between return light from the subject's eye and the reference light is acquired. A method for generating the pseudo image of the fundus is such that information in the depth direction (Z direction) of the fundus acquired by subjecting the interference signal, for example, to signal processing is converted into density or luminance to image the state of a fundus surface layer. The display control unit 120B superimposes the region of interest on the pseudo fundus image input to the imaging control unit 120A to generate the synthesized fundus image and displays the synthesized fundus image together with the tomographic image on the display unit 130.

As described above, according to the present exemplary embodiment, the fundus camera for capturing the fundus image is not mounted on the apparatus. The area for capturing the tomographic image can be grasped by generating the pseudo fundus image in light interference signal processing and displaying the region of interest superimposed on the fundus image.

In the first exemplary embodiment as described above, an amount of displacement of the fundus of the subject's eye 100 is calculated from the fundus image to track the involuntary movement of the subject's eye. In a ninth exemplary embodiment, on the other hand, an example will be described in which the involuntary movement is tracked by a displacement amount of the subject's anterior eye 100.

The configuration of the ophthalmologic apparatus according to the present exemplary embodiment is described below with the imaging unit 110 and the fundus tracking unit 150 in FIG. 1 as an imaging unit 110C and an anterior eye tracking unit 150C respectively.

[Function of Imaging Unit 110C]

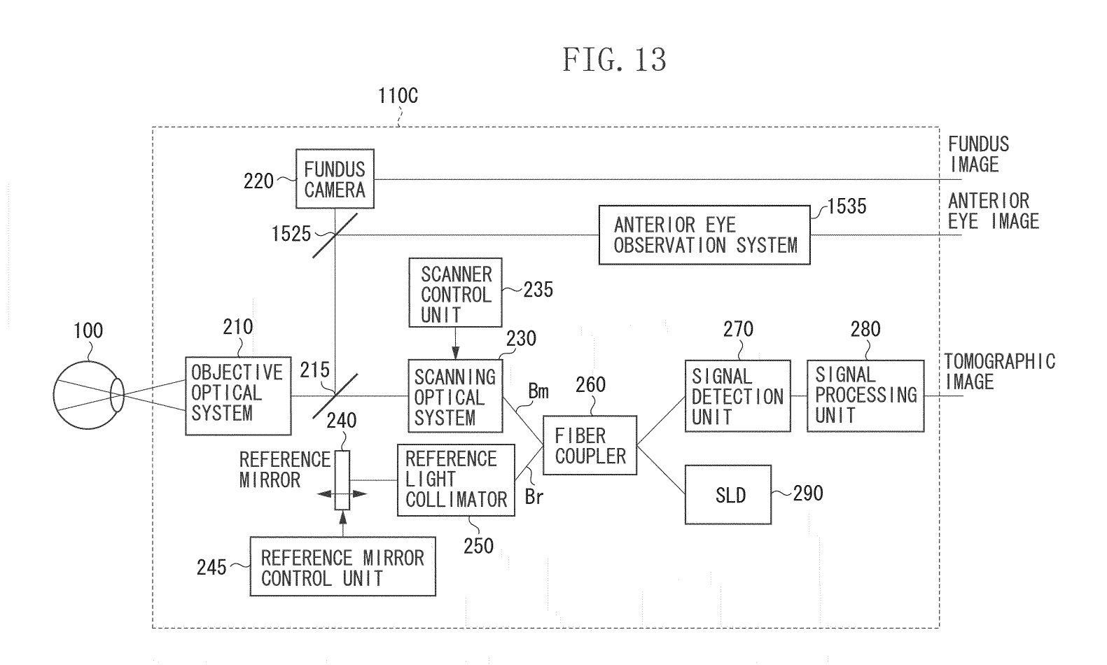

A specific example of the imaging unit 110C according to the present exemplary embodiment is described below with reference to FIG. 13.

In the imaging unit 110C according to the present exemplary embodiment, a dichroic mirror 1525 and an anterior eye observation system 1535 are added to the imaging unit 110 according to the first exemplary embodiment. The other components are similar in function to those of the first exemplary embodiment.

The anterior eye observation system 1535 includes an illumination light source for observing the anterior eye, a lens, and an infrared charge coupled device (CCD). The infrared CCD shows sensitivity in the vicinity of a wavelength of the illumination light for observing the anterior eye, specifically, in the vicinity of 780 nm. The dichroic mirror 1525, as its characteristic, reflects infrared light in the vicinity of a wavelength of the illumination light for observing the anterior eye and infrared light in other wavelength regions penetrates the dichroic mirror 1525. In the above configuration, the subject's eye 100 is irradiated with the illumination light for observing the anterior eye and the reflected light by the anterior eye part is caused to pass through the objective optical system 210, reflected by the half mirror 215 and the dichroic mirror 1525, and guided to the infrared CCD. Thus, the anterior eye image of the subject's eye 100 can be captured. In other words, the anterior eye observation system 1535 corresponds to one example of an anterior eye acquisition unit for acquiring an anterior-eye image of the subject's eye.

[Function of Anterior Eye Tracking Unit 150C]

The anterior eye tracking unit 150C analyzes, for example, the momentum of an iris or a pupil, from the anterior-eye image captured by the imaging unit 110C to calculate an amount of displacement by using a calculation method similar to the one used in the first exemplary embodiment. In other words, the anterior eye tracking unit 150C is an example of a displacement acquisition unit for acquiring displacement of an image capturing position between the anterior-eye images acquired by the anterior eye acquisition unit.

If the anterior-eye image is subjected to a binary processing using image processing, there is an advantage that an iris or a pupil is high in contrast and easy to identify.

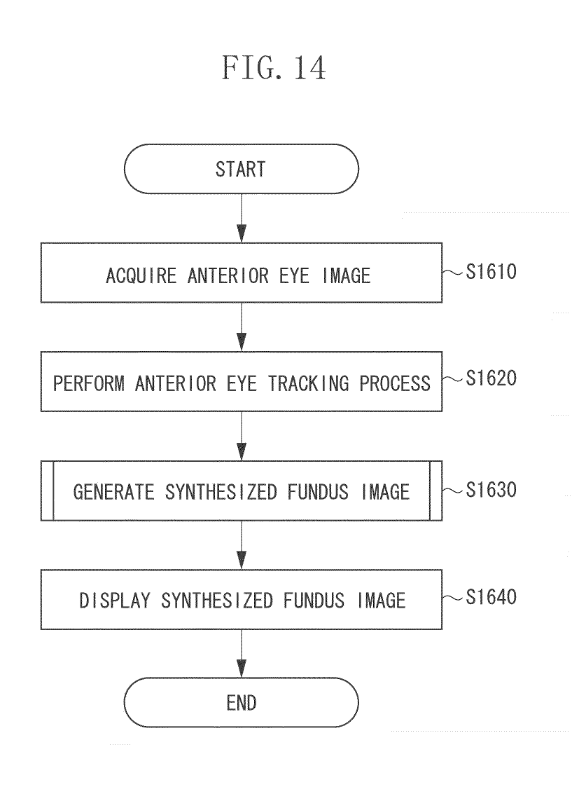

A procedure of a specific processing executed by the ophthalmologic apparatus 10 according to the present exemplary embodiment is described below with reference to a flow chart in FIG. 14.

In step S1610, the imaging control unit 120A outputs a command to capture the anterior-eye image and the fundus image to the imaging unit 110C to acquire the anterior-eye image and the fundus image of the subject's eye 100 captured by the imaging unit 110C. The imaging control unit 120A outputs the anterior-eye image to the anterior eye tracking unit 150C and the fundus image to the display control unit 120B. Further, the imaging control unit 120A outputs the imaging control information used for capturing the tomographic image to the display control unit 120B.

In step S1620, the anterior eye tracking unit 150C tracks the anterior eye to calculate the amount of displacement between the anterior-eye images.

In step S1630, the display control unit 120B generates a synthesized fundus image by superimposing the region of interest indicating the position where the tomographic image is captured, on the fundus image. In the present exemplary embodiment, the display control unit 120B generates the synthesized fundus image after correcting the position where a frame indicating the region of interest is superimposed on the fundus image to decrease the displacement based on the amount of displacement of the fundus image calculated in step S1620.

In step S1640, the display unit 130 displays the synthesized fundus image generated in step S1630.

As described above, according to the present exemplary embodiment, the anterior-eye image for tracking the involuntary movement of the subject's eye is utilized, which shows contrast higher than when the fundus image is utilized so that accuracy in calculating the amount of displacement of the subject's eye is improved.

The third exemplary embodiment has described the example in which the movement of the region of interest is stopped if the index moving on the display unit 130 is positioned on the fundus image displayed on the display unit 130. That is a display control method for reducing the motion of the region of interest when the operator operates the region of interest. On the other hand, a tenth exemplary embodiment will describe an example in which, if the index is positioned on the fundus image and does not move for a certain period of time, the region of interest is moved based on the calculated displacement.

The ophthalmologic apparatus 10 of the present exemplary embodiment is similar in configuration to that of the first exemplary embodiment, so that the description of the configuration is omitted. Processing according to the present exemplary embodiment is similar to the processing illustrated by the flow chart in FIG. 7 according to the third exemplary embodiment except step S730, so that the description thereof is omitted. Step S730 executed in the present exemplary embodiment is described as step S730B.

In step S730B, the display control unit 120B generates a synthesized fundus image by superimposing the region of interest indicating the position where the tomographic image is captured, on the fundus image. More specifically, the display control unit 120B acquires the position of an index moving on the display unit 130 according to the instruction of the operation unit 140 to determine whether the index exists on the fundus image displayed on the display unit 130. If the index is not positioned on the fundus image, the region of interest is moved on display unit based on the calculated displacement. On the other hand, if the index is positioned on the fundus image, the movement of the region of interest is stopped. If the index is positioned on the fundus image but does not move for a certain period of time (e.g., several seconds), the region of interest is moved based on the calculated displacement. In other words, if the index is positioned on the fundus image for a certain period of time or longer, the movement of the region of interest is resumed.

As described above, according to the present exemplary embodiment, even if the index is positioned on the fundus image, by determining the operation state of the operation unit, occurrence of a malfunction in which the movement of the region of interest is stopped contrary to the operator's intension, is reduced.

The present invention is not limited to the above exemplary embodiments. It is to understand that various modifications and changes may be made without departing from the gist of the present invention. For example, the above exemplary embodiments may be combined.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-017617 filed Jan. 31, 2013, which is hereby incorporated by reference herein in its entirety.

What is claimed is:

1. An ophthalmologic apparatus comprising:
an acquisition unit configured to acquire a fundus image of a subject's eye;
a displacement acquisition unit configured to acquire a displacement of an imaging position between the fundus images acquired by the acquisition unit; and
a display control unit configured to display on a display unit the fundus image acquired by the acquisition unit and a region of interest which is an area where a tomogram of the fundus is taken;
wherein the display control unit displays the fundus image and the region of interest on the display unit based on the displacement acquired by the displacement acquisition unit so that the region of interest is positioned at a predetermined position of the fundus image, and displays the region of interest on the display unit by changing a size of the region of interest according to a range where the fundus image is captured.

2. The ophthalmologic apparatus according to claim 1, wherein, in a case where the size of a fundus image display area on the display unit is fixed, the larger the range where the fundus image is captured, the smaller the size of the region of interest.

3. The ophthalmologic apparatus according to claim 1, wherein, in a case where the size of the fundus image display area on the display unit is variable according to the range where the fundus image is captured, the larger the range where the fundus image is captured, the larger the size of the fundus image display area and the region of interest.

4. An ophthalmologic apparatus comprising:
an acquisition unit configured to acquire a fundus image acquired based on interference light between return light from a subject's eye and reference light;
a displacement acquisition unit configured to acquire the displacement of an imaging position between the fundus images acquired by the acquisition unit; and
a display control unit configured to display on a display unit the fundus image acquired by the acquisition unit and a region of interest which is an area where a tomogram of the fundus is taken;
wherein the display control unit displays the fundus image and the region of interest on the display unit based on the displacement acquired by the displacement acquisition unit so that the region of interest is positioned at a predetermined position of the fundus image.

5. An ophthalmologic apparatus comprising:
an acquisition unit configured to acquire a fundus image of a subject's eye;
an anterior-eye acquisition unit configured to acquire an anterior-eye image of the subject's eye;
a displacement acquisition unit configured to acquire a displacement of an imaging position between the anterior-eye images acquired by the anterior-eye acquisition unit; and
a display control unit configured to display on a display unit the fundus image acquired by the acquisition unit and a region of interest which is an area where a tomogram of the fundus is taken;
wherein the display control unit displays the fundus image and the region of interest on the display unit based on the displacement acquired by the displacement acquisition unit so that the region of interest is positioned at a predetermined position of the fundus image.

6. The ophthalmologic apparatus according to claim 1, wherein the display control unit moves the region of interest on the display unit based on the displacement acquired by the displacement acquisition unit to display the region of interest at the predetermined position on the fundus image.

7. The ophthalmologic apparatus according to claim 1, wherein the display control unit displays the fundus image on the display unit in such a way as to reduce the displacement based on the displacement acquired by the displacement acquisition unit to display the region of interest at the predetermined position on the fundus image.

8. The ophthalmologic apparatus according to claim 6, further comprising an operation unit configured to receive operation from an inspector,
wherein the display control unit displays the fundus image in a part of the area of the display unit, indicates any position of the display unit in an area on the display unit other than the part of the area, and moves the region of interest on the display unit based on the displacement acquired by the displacement acquisition unit if there is an index movable according to the operation of the operation unit but stops moving the region of interest if there is the index on the fundus image.

9. The ophthalmologic apparatus according to claim 8, wherein the display control unit moves the region of interest on the display unit based on the displacement acquired by the displacement acquisition unit if there is the index in an area on the display unit other than the region of interest but stops moving the region of interest if there is the index in the region of interest.

10. The ophthalmologic apparatus according to claim 9, wherein the display control unit resumes moving of the region of interest if there is the index in the region of interest for a predetermined period of time or longer.

11. The ophthalmologic apparatus according to claim 6, further comprising an operation unit configured to receive operation from an inspector,
wherein the display control unit displays the fundus image in a part of the area of the display unit, indicates any position of the display unit in an area on the display unit other than the part of the area, displays the region of interest at the predetermined position on the fundus image by moving the region of interest on the display unit based on the displacement acquired by the displacement acquisition unit if there is an index movable according to the operation of the operation unit, and also displays the fundus image on the display unit in such a way as to reduce the displacement based on the displacement acquired by the displacement acquisition unit if there is the index on the fundus image.

12. The ophthalmologic apparatus according to claim 11, wherein the display control unit displays the region of interest at the predetermined position on the fundus image by moving the region of interest on the display unit based on the displacement acquired by the displacement acquisition unit if there is the index in an area on the display unit other than the region of interest, and displays the fundus image on the display unit in such a way as to reduce the displacement based on the displacement acquired by the displacement acquisition unit if there is the index in the region of interest.

13. The ophthalmologic apparatus according to claim 8, wherein the position and size of the region of interest can be changed according to the operation of the operation unit.

14. An ophthalmologic apparatus comprising:
an acquisition unit configured to acquire a fundus image of a subject's eye;
a displacement acquisition unit configured to acquire the displacement of an imaging position between the fundus images acquired by the acquisition unit; and
a display control unit configured to display the fundus image acquired by the displacement acquisition unit and a region of interest which is an area where a tomogram of the fundus is taken on a display unit so that the region of interest is positioned in a predetermined position of the fundus image based on the displacement acquired by the displacement acquisition unit;
wherein the display control unit displays a common portion between the fundus images on the display unit in such a way as to reduce the displacement based on the displacement acquired by the displacement acquisition unit to display the region of interest in the predetermined position on the fundus image, and enlarges and displays the region of interest and the common portion.

15. An ophthalmologic apparatus comprising:
an acquisition unit configured to acquire a fundus image of a subject's eye;
a displacement acquisition unit configured to acquire a displacement of an imaging position between the fundus images acquired by the acquisition unit;
a display control unit configured to display the fundus image acquired by the displacement acquisition unit and a region of interest which is an area where a tomogram of the fundus is taken on a display unit so that the region of interest is positioned in a predetermined position of the fundus image based on the displacement acquired by the displacement acquisition unit; and
an operation unit configured to receive operation from an inspector;
wherein the display control unit moves the region of interest on the display unit based on the displacement acquired by the displacement acquisition unit to display the region of interest in the predetermined position and indicates any position of the display unit in an area on the display unit other than the region of interest, moves the region of interest on the display unit based on the displacement acquired by the displacement acquisition unit if there is an index movable according to the operation of the operation unit, stops moving of the region of interest if there is the index in the region of interest, and resumes moving of the region of interest if there is the index in the region of interest for a predetermined time or longer.

16. An ophthalmologic apparatus comprising:
an acquisition unit configured to acquire a fundus image of a subject's eye;
a displacement acquisition unit configured to acquire a displacement of an imaging position between the fundus images acquired by the acquisition unit; and
a display control unit configured to display the fundus image acquired by the acquisition unit and a region of interest which is an area where a tomogram of the fundus is taken on a display unit;
wherein the display control unit performs display on the display unit by retaining a positional relationship between the fundus image and the region of interest based on the displacement acquired by the displacement acquisition unit.

17. A method for controlling an ophthalmologic apparatus including an acquisition unit, a displacement acquisition unit, and a display control unit, the method comprising:
acquiring a fundus image of a subject's eye with the acquisition unit;
acquiring the displacement of an imaging position between the acquired fundus images with the displacement acquisition unit; and
displaying the acquired fundus image and a region of interest which is an area where a tomogram of the fundus is taken on a display unit, with the display control unit;
wherein in the displaying the display control unit displays the fundus image and the region of interest on the display unit based on the acquired displacement so that the region of interest is positioned at a predetermined position of the fundus image, and displays the region of interest on the display unit by changing a size of the region of interest according to a range where the fundus image is taken.

18. A non-transitory storage medium storing a program for causing a computer to execute each step of a method for controlling an ophthalmologic apparatus according to claim 17.
